(19) United States
(12) Patent Application Publication
(10) Pub. No.: US 2015/0355204 A1
(43) Pub. Date: Dec. 10, 2015

(54) MARKER FOR DEPRESSION, ASSAY METHOD, METHOD FOR DETERMINING DEPRESSION, SCREENING METHOD FOR ANTIDEPRESSANTS, AND KIT

(71) Applicant: MEIJO UNIVERSITY, Aichi (JP)

(72) Inventors: Toshitaka NABESHIMA, Nagoya-shi, Aichi (JP); Akihiro MOURI, Nagoya-shi, Aichi (JP); Yukihiro NODA, Nagoya-shi, Aichi (JP)

(73) Assignee: MEIJO UNIVERSITY, Nagoya-shi, Aichi (JP)

(21) Appl. No.: 14/760,839

(22) PCT Filed: Jan. 17, 2014
(86) PCT No.: PCT/JP2014/050746

§ 371 Date: Jul. 14, 2015

(30) Foreign Application Priority Data
Jan. 17, 2013 (JP) 2013-005911

(51) Int. Cl. G01N 33/94

(52) U.S. Cl. CPC G01N 33/9433; G01N 33/9413; G01N 2440/36; G01N 2440/14; G01N 2333/4703; G01N 2800/28

(57) ABSTRACT

Disclosed herein are a method and a kit using a novel marker associated with depression. The marker for depression includes one or more selected from a noradrenaline transporter and a dopamine transporter. The method for determining depression includes a step of examining an expression level of the marker for depression in a blood sample collected from a subject.

### TECHNICAL FIELD

The present invention relates to a method and a kit using a novel marker associated with depression.

### BACKGROUND ART

In Diagnostic and Statistical Manual of Mental Disorders, Fourth Edition (DSM-4) by American Psychiatric Association, depression is defined as a disorder with symptoms such as chronic depressed mood, loss of interest or pleasure, significant body weight change, insomnia or hypersomnia, loss of energy, psychomotor agitation or retardation, feelings of worthlessness or guilt, diminished ability to think or concentrate, and suicidal thoughts.

The monoamine hypothesis has been proposed in which reduced neurotransmission of serotonin, noradrenaline, and dopamine is associated with the pathology of depression. As for noradrenaline, signaling in healthy subjects is mediated by the release of a sufficient amount of noradrenaline from the presynaptic nerve terminal into the synaptic cleft and the reception of noradrenaline by the noradrenaline receptor present in the postsynaptic nerve terminal, and excessive noradrenaline in the synaptic cleft is removed by the noradrenaline transporter so that noradrenaline is again released from the presynaptic nerve terminal into the synaptic cleft.

On the other hand, it is considered that in patients with depression, the amount of noradrenaline released from the presynaptic nerve terminal is insufficient, and therefore the postsynaptic nerve terminal cannot receive sufficient noradrenaline. That is, patients with depression are considered to be deficient in noradrenaline in the synaptic cleft.

Based on the monoamine hypothesis, methods of diagnosing depression using noradrenaline as an indicator and antidepressants containing, as an active ingredient, a noradrenaline reuptake inhibitor, such as desipramine, have been proposed.

It has been reported that the noradrenaline transporter is expressed in the central nervous system and in the periphery such as leukocytes. The noradrenaline transporter in leukocytes has many properties in common with the noradrenaline transporter in central noradrenergic neurons. Therefore, studies have been made to apply leukocytes, including lymphocytes, obtained from peripheral blood as a biomarker for depression.

Further, it has been reported that there is a relationship between a change in noradrenaline transporter binding affinity and the ratio between noradrenaline and its metabolite and psychological symptoms such as depression. In this report, noradrenaline transporter binding affinity was examined based on binding of a drug that acts on the noradrenaline transporter. However, a mechanism supporting the relationship is still unknown.

Further, many animal models of depression have been proposed to develop antidepressants for treating noradrenaline-related depression. These animal models exhibit depression-like behavior, and stop exhibiting depression-like behavior by inhibition of reuptake of noradrenaline from the synaptic cleft. For this reason, the animal models of depression are used for the screening of candidates for antidepressants.

### PRIOR ART DOCUMENTS

### Non-Patent Documents

Non-Patent Document 1: Bruss M, Kunz J, Lingen B, Bonisch H. Chromosomal mapping of the human gene for the tricyclic antidepressant-sensitive noradrenaline transporter. Hum Genet. 1993 April; 91(3): 278-80
Non-Patent Document 1 discloses that noradrenaline transporters in the brain and periphery are derived from the same gene.
Non-Patent Document 2: Klimek V, Stockmeier C, Overholser J, Meltzer H Y, Kalka S, Dilley G, Ordway G A. Reduced levels of norepinephrine transporters in the locus coeruleus in major depression. J Neurosci. 1997 November 1; 17(21): 8451-8
Non-Patent Document 2 discloses that changes in noradrenaline transporter expression are observed in the brain of patients with depression after death from suicide.
Non-Patent Document 3: Mata S, Urbina M, Manzano E, Ortiz T, Lima L. Noradrenaline transporter and its turnover rate are decreased in blood lymphocytes of patients with major depression. J Neuroimmunol. 2005 December 30; 170(1-2): 134-40
Non-Patent Document 3 discloses that changes in noradrenaline transporter binding affinity and the ratio between noradrenaline and its metabolite are observed in lymphocytes of patients with depression.
Non-Patent Document 4: Jayanthi L D, Annamalai B, Samuvel D J, Gether U, Ramamoorthy S. Phosphorylation of the norepinephrine transporter at threonine 258 and serine 259 is linked to protein kinase C-mediated transporter internalization. J Biol Chem. 2006 August 18; 281(33): 23326-40
Non-Patent Document 4 discloses that phosphorylation of the noradrenaline transporter reduces the abundance of the noradrenaline transporter on the cell membrane surface and the activity of the noradrenaline transporter.

### SUMMARY OF THE INVENTION

### Problems to be Solved by the Invention

Depression can be diagnosed from the behavior of a patient, but can also be physically, chemically, or biologically diagnosed by using a specific marker.

A method of diagnosing depression has been previously disclosed, which uses the amount of noradrenaline in blood as a marker based on the monoamine hypothesis. However, the accuracy of the diagnostic method is not necessarily satisfactory. This is because noradrenaline in the body is present also in the adrenal medulla and in sympathetic nerves, and therefore the amount of noradrenaline in blood poorly reflects the amount of noradrenaline in the brain.

Further, a method of diagnosing depression by visualizing the noradrenaline transporter in the brain has also been disclosed. However, this method needs an expensive apparatus and the administration of a radioactive tracer to the human body. For this reason, there has been a demand for a method for more simply diagnosing depression.

The present inventors have studied the physiological functions of MAGE-D1 gene-deficient mice by using them. As a result, the present inventors have found that MAGE-D1 gene-deficient mice exhibit depression-like behavior, and stop exhibiting depression-like behavior by giving these mice a selective noradrenaline reuptake inhibitor that is effective for humans. That is, the present inventors have found that these mice are a very valid model of noradrenaline-related human depression. MAGE-D1 gene-deficient mice are considered to have the same mechanism of depression as humans, because these mice exhibit human depression-like behavior, and stop exhibiting depression-like behavior by giving them an antidepressant for humans.

The present inventors have found that the expression level of the noradrenaline transporter in lymphocytes derived from peripheral blood of patients with depression is higher than that of healthy subjects.

As a result of further study of the mechanism of noradrenaline-related depression based on the above findings, the present inventors have identified the ubiquitinated noradrenaline transporter. Further, it has been reported that the function of the noradrenaline transporter is reduced by phosphorylation.

From the above results obtained using MAGE-D1 gene-deficient mice and lymphocytes derived from patients with depression, it is considered that the expression level or function of the noradrenaline transporter is regulated by addition of ubiquitin or phosphoric acid and this flow is regulated by MAGE-D1 protein.

Based on the above findings, the present inventors have completed the present invention. It is an object of the present invention to provide a method and a kit using a novel marker associated with depression.

### Solutions to the Problems

(First Aspect)

In order to achieve the above object, a first aspect of the present invention provides a marker for depression, comprising one or more selected from a noradrenaline transporter and a dopamine transporter.

(Second Aspect)

In order to achieve the above object, a second aspect of the present invention provides a method for determining depression, comprising a step of examining an expression level of the marker for depression according to the first aspect in a blood sample collected from a subject.

In this application, the expression level of the marker means the amount of a translation product. That is, the marker can be measured or analyzed at the level of protein.

(Third Aspect)

In order to achieve the above object, a third aspect of the present invention provides the method for determining depression according to the second aspect, further comprising the step of comparing the expression level of the marker for depression according to a first aspect in the blood sample collected from the subject with an expression level of the marker for depression according to the first aspect obtained as a control from at least one healthy subject.

(Fourth Aspect)

In order to achieve the above object, a fourth aspect of the present invention provides the method for determining depression according to the third aspect, wherein the subject is determined to have depression when the expression level of the marker for depression according to the first aspect in the blood sample collected from the subject is at a higher level than the control as a result of the comparison.

(Fifth Aspect)

As described above, the present inventors have identified the ubiquitinated noradrenaline transporter. Further, it has been reported that the function of the noradrenaline transporter is reduced by phosphorylation. It is reasonably assumed that, also in humans, the activity of the noradrenaline transporter is regulated by transfer from the cell membrane surface into the cell caused by addition of phosphoric acid, and the molecular weight of the noradrenaline transporter is increased by addition of ubiquitin and the ubiquitinated noradrenaline transporter is degraded by proteasome.

In order to achieve the above object, a fifth aspect of the present invention provides a method for determining depression, comprising a step of analyzing the following (1) and (2) in a blood sample collected from a subject:

(1) a noradrenaline transporter expression level; and

(2) a ratio of a ubiquitinated and/or phosphorylated noradrenaline transporter.

(Sixth Aspect)

In order to achieve the above object, a sixth aspect of the present invention provides the method for determining depression according to the fifth aspect, further comprising a step of comparing analysis results of the following (3) and (4) in the subject with analysis results of the following (3) and (4) obtained as controls from at least one healthy subject:

(3) a noradrenaline transporter expression level; and

(4) a ratio of a ubiquitinated and/or phosphorylated noradrenaline transporter.

(Seventh Aspect)

In order to achieve the above object, a seventh aspect of the present invention provides the method for determining depression according to the sixth aspect, wherein the subject is determined to have depression when the noradrenaline transporter expression level obtained from the blood sample collected from the subject is at a higher level than the control or when the ratio of the ubiquitinated and/or phosphorylated noradrenaline transporter obtained from the blood sample collected from the subject is at a lower level than the control as a result of the comparison.

(Eighth Aspect)

In order to achieve the above object, an eighth aspect of the present invention provides a kit comprising a detector of the marker for depression according to the first aspect.

(Ninth Aspect)

In order to achieve the above object, a ninth aspect of the present invention provides the kit according to the eighth aspect, further comprising one or more selected from a ubiquitinated protein collector and a phosphorylated protein collector.

(Tenth Aspect)

In order to achieve the above object, a tenth aspect of the present invention provides an assay method for assisting diagnosis of depression, comprising a step of examining an expression level of the marker for depression according to the first aspect.

(Eleventh Aspect)

In order to achieve the above object, an eleventh aspect of the present invention provides an assay method for assisting diagnosis of depression, comprising a step of examining ubiquitination and/or phosphorylation of the marker for depression according to the first aspect.

(Twelfth Aspect)

In order to achieve the above object, a twelfth aspect of the present invention provides a screening method for antidepressants, comprising a step of examining a change in an expression level of the marker for depression according to the first aspect.

### Effects of the Invention

As described above, it is reasonably assumed that also in the case of humans, an increase in noradrenaline transporter expression level can be an indicator of depression. Further, it is reasonably assumed that the molecular weight of the noradrenaline transporter is increased by addition of ubiquitin and the ubiquitinated noradrenaline transporter is degraded by proteasome. Further, it is reasonably assumed that the function of the noradrenaline transporter is reduced by addition of phosphoric acid.

The previous invention disclosed by the present inventors in PCT/JP2012/068348 focuses attention on the relationship between the serotonin transporter and ubiquitination thereof and depression.

From the above, it is suggested that the expression level or ubiquitination of the serotonin transporter and the noradrenaline transporter is associated with depression. It is assumed that the dopamine transporter is also associated with depression in light of the monoamine hypothesis.

Therefore, the first aspect provides a useful marker for depression. The marker for depression can be preferably used for, for example, one or more selected from an assay for assisting the diagnosis of depression, diagnosis of depression, determination of depression, and screening of antidepressants.

Further, it is reasonably assumed that it is useful to use the ubiquitination and/or phosphorylation ratio of the marker for depression according to the first aspect. The fifth to seventh aspects provide a method for determining depression, which uses the expression level of the noradrenaline transporter as a novel marker and the ratio of the ubiquitinated and/or phosphorylated noradrenaline transporter.

Based on the facts found by the present inventors, it is considered that if the noradrenaline transporter is not ubiquitinated nor phosphorylated, the noradrenaline transporter is excessively present in the body, and therefore the amount of noradrenaline in the synaptic cleft is insufficient so that depression is caused. Further, as described above, it has been reported that there is a relationship between the expression of the noradrenaline transporter in the central nervous system and the expression of the noradrenaline transporter in platelets and leukocytes including lymphocytes.

The method according to the fifth to seventh aspects uses the expression level of the noradrenaline transporter and the ratio of the ubiquitinated and/or phosphorylated noradrenaline transporter of the same sample. Therefore, it is considered that the method is less influenced by conditions during blood sample collection (e.g., change in physical conditions, difference in the amount of a sample collected, difference in the timing of sample collection) or individual difference, and therefore gives a highly accurate result.

It is considered that the noradrenaline transporter is degraded by proteasome after being ubiquitinated. Therefore, it is considered that the ratio of the ubiquitinated noradrenaline transporter obtained by analyzing a blood sample includes information corresponding to the result of measuring a change in the amount of noradrenaline not only at the time of blood sample collection but also for a certain period of time.

Further, it is considered that the activity of the noradrenaline transporter is reduced by transfer from the cell membrane surface into the cell after being phosphorylated. Therefore, it is considered that the ratio of the phosphorylated noradrenaline transporter obtained by analyzing a blood sample includes information corresponding to the result of measuring a change in the amount of noradrenaline not only at the time of blood sample collection but also for a certain period of time.

The degradation pathway of the noradrenaline transporter through ubiquitination and the activity regulatory pathway of the noradrenaline transporter through phosphorylation have been previously unknown, and therefore it has been difficult to predict even a change in the amount of noradrenaline in blood. Further, continuous blood sampling from a subject places a heavy burden on the body of the subject, and therefore the diagnosis of depression has been based on limited information about the amount of noradrenaline in blood only at the time of blood sample collection. Further, as described above, noradrenaline in the body is present also in the sympathetic nervous system, adrenal medulla, etc., and therefore it is considered that the amount of noradrenaline in blood poorly reflects the amount of noradrenaline in the brain.

On the other hand, when attention is focused not on noradrenaline but on the noradrenaline transporter, the noradrenaline transporter in platelets and leukocytes including lymphocytes has many properties in common with the noradrenaline transporter in central noradrenergic neurons. It is considered that the method using the marker for depression according to the present invention disclosed in this application takes more information with higher quality into account and therefore achieves higher accuracy as compared to the method of diagnosing depression using the amount of noradrenaline in blood at the time of blood sample collection as a marker.

Further, the method according to the present invention does not require an expensive visualization apparatus or the administration of a radioactive tracer to the human body, and is therefore a simple diagnostic method that is less burdensome to the human body as compared to a method of diagnosing depression by visualizing the noradrenaline transporter in the brain.

The method for determining depression becomes more effective by comparing the ratio of the ubiquitinated and/or phosphorylated noradrenaline transporter obtained from a blood sample collected from a subject with the ratio of the ubiquitinated and/or phosphorylated noradrenaline transporter obtained as a control from a healthy subject.

The method for determining depression becomes even more effective by determining a subject to have depression when the expression level of the noradrenaline transporter obtained from a blood sample collected from the subject is higher than a control or when the ratio of the ubiquitinated and/or phosphorylated noradrenaline transporter obtained from a blood sample collected from the subject is lower than a control.

The eighth and ninth aspects provide a useful kit. The composition of the kit is based on the finding, obtained by the present inventors, that “it is considered that the molecular weight of the marker for depression disclosed in this application is increased by addition of ubiquitin and the ubiquitinated marker for depression is degraded by proteasome, and the activity of the marker for depression is reduced by transfer from the cell membrane surface into the cell after being phosphorylated”. Particularly, the kit has a beneficial effect when used in the diagnosis of depression. Further, the kit is also useful for use in carrying out the invention using the marker for depression disclosed in this application.

### BRIEF DESCRIPTION OF THE DRAWINGS

FIG. 1 shows that depression-like behavior in model mice is relieved by a human antidepressant, desipramine and an anti-attention deficit hyperactivity disorder (ADHD) medication, atomoxetine that have a noradrenaline transporter inhibitory effect.

FIG. 2 shows that noradrenaline contents in the frontal cortex, hippocampus, and amygdala of the brain of model mice are significantly reduced.

FIG. 3 shows that an increase in the amount of released extracellular noradrenaline caused by high-potassium stimulation is significantly reduced in the frontal cortex and hippocampus of model mice.

FIG. 4(a) shows that the amount of noradrenaline transporter protein is increased in the frontal cortex of model mice, FIG. 4(b) shows that there is no significant difference in the amount of noradrenaline transporter mRNA between such model mice and control mice, and FIG. 4(c) shows that the expression of ubiquitinated noradrenaline transporter protein is observed in mice, but there is no significant difference in the amount of ubiquitinated noradrenaline transporter between model mice and control mice.

FIG. 5 shows the expression level of noradrenaline transporter in established lymphocytes derived from antidepressant-responsive patients with depression was significantly higher than that derived from healthy subjects.

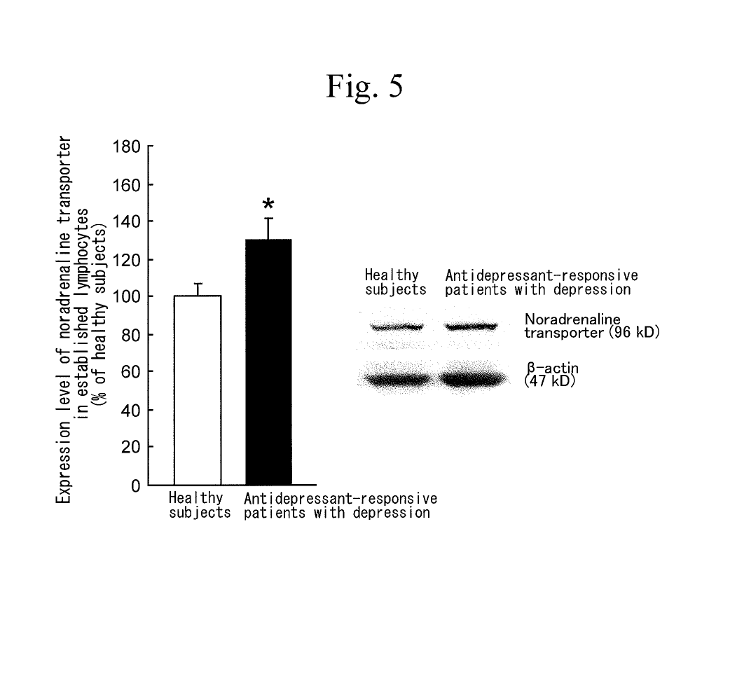

### EMBODIMENTS OF THE INVENTION

Hereinbelow, embodiments for carrying out the present invention, including the best embodiment, will be described.

In the present invention, ubiquitin is not particularly limited as long as it is used for protein degradation in the ubiquitin-dependent proteasome system.

In the present invention, phosphoric acid is not particularly limited as long as it is used for modification of the noradrenaline transporter or the dopamine transporter.

In the present invention, a marker for depression including the noradrenaline transporter and/or the dopamine transporter is not particularly limited as long as the noradrenaline transporter participates in noradrenaline uptake and the dopamine transporter participates in dopamine uptake. The marker for depression is preferably the noradrenaline transporter.

The noradrenaline transporter includes, for example, wild-type (GenBank Accession No.: AK312793.1) and exon 6-deficient polymorphisms. Noradrenaline transporters with various molecular weights can be detected in a sample prepared from, for example, platelets or leukocytes including lymphocytes by Western blotting.

As the dopamine transporter, for example, wild-type (GenBank Accession No.: NM001044.4) is known.

Further, the marker for depression is preferably one expressed in neurons forming the synaptic cleft, one present in platelets, one present in leukocytes including lymphocytes, one present in sympathetic nerves of each peripheral tissue, or one present in the adrenal medulla, and is more preferably one expressed in neurons forming the synaptic cleft, one present in platelets, or one present in leukocytes including lymphocytes, and is even more preferably one present in platelets or one present in lymphocytes.

In the present invention, the ubiquitinated marker for depression is one combined with one or more molecules of ubiquitin. Ubiquitin is a low-molecular-weight protein having a molecular weight of about 8.6 kD. For example, the ubiquitinated noradrenaline transporter shows a smeared high molecular weight signal starting from the original molecular weight (about 80 kD) in electrophoresis or the like, and is preferably one having a molecular weight of about 100 kD or more.

It is reasonably assumed that also in humans, the molecular weight of the marker for depression is increased by addition of ubiquitin, and the ubiquitinated marker for depression is degraded by proteasome. Further, it is considered that if the marker for depression is not ubiquitinated, the marker for depression is excessively present in the body, and therefore the amount of, for example, noradrenaline in the synaptic cleft is insufficient so that depression is caused. From this, it is considered that when the ratio of the ubiquitinated marker for depression is lower, the possibility of having depression is higher.

In the present invention, the ratio of the ubiquitinated marker for depression indicates the ratio of the ubiquitinated marker for depression in a sample. A formula for calculating the ratio can be appropriately selected, and examples of the formula include: (A) the amount of the ubiquitinated marker for depression relative to the total amount of the marker for depression; (A′) the total amount of the marker for depression relative to the amount of the ubiquitinated marker for depression; (B) the amount of the ubiquitinated marker for depression relative to the amount of the marker for depression not combined with ubiquitin; (B′) the amount of the marker for depression not combined with ubiquitin relative to the amount of the ubiquitinated marker for depression; (C) the amount of the high-molecular-weight-gained marker for depression relative to the total amount of the marker for depression; (C′) the total amount of the marker for depression relative to the amount of the high-molecular-weight-gained marker for depression; (D) the amount of the high-molecular-weight-gained marker for depression relative to the amount of the marker for depression not combined with ubiquitin; and (D′) the amount of the marker for depression not combined with ubiquitin relative to the amount of the high-molecular-weight-gained marker for depression. Depending on the selection of the calculating formula, there is a case where the higher value of the ratio means that the ratio of the ubiquitinated marker for depression is lower. When the ratio of a subject is compared with a control obtained from a healthy subject, a method for calculating the ratio of the subject is preferably the same as a method for calculating the control.

The embodiments of the present invention include the examination of a change in the ratio of the ubiquitinated marker for depression more markedly in a sample in which the degradation of the ubiquitinated marker for depression with proteasome is inhibited by a proteasome inhibitor.

In the present invention, the ratio of the phosphorylated marker for depression indicates the ratio of the phosphorylated marker for depression in a sample. A formula for calculating the ratio can be appropriately selected, but may be selected from, for example, calculating formulas similar to the calculating formulas (A) to (D′) described above with reference to the ratio of the ubiquitinated marker for depression. Depending on the selection of the calculating formula, there is a case where the higher value of the ratio means that the ratio of the phosphorylated marker for depression is lower. When the ratio of a subject is compared with a control obtained from a healthy subject, a method for calculating the ratio of the subject is preferably the same as a method for calculating the control.

The phrase “ubiquitinated and/or phosphorylated” includes a case where the marker for depression is ubiquitinated, a case where the marker for depression is phosphorylated, and a case where the marker for depression is ubiquitinated and phosphorylated. The ubiquitination ratio of the marker for depression or the phosphorylation ratio of the marker for depression can be appropriately used. That is, both the ratios may be used or one of the ratios may be appropriately used.

A unit used in the calculation of the amount of the marker for depression or the ratio is not particularly limited, and may be appropriately selected according to a method for analyzing a sample. For example, mass, the number of molecules, or signal intensity by electrophoresis including Western blotting may be used.

Examples of a quantitative method includes immunological methods such as Western blotting, flow cytometry, ELISA, EIA, RIA, FIA, chemiluminescent immunoassay, and ECLIA. As another quantitative method, a known method such as a method using electrophoresis, a method using absorbance, or a method using a quartz oscillation biosensor can be appropriately used. The method preferably uses signal intensity by Western blotting. The method more preferably uses mass or the number of molecules by ELISA (Enzyme-Linked ImmunoSorbent Assay), EIA (Enzyme ImmunoAssay), or RIA (Radio-ImmunoAssay). When a unit whose value varies depending on the degree of ubiquitination or phosphorylation, such as mass, is used, for example, a converted value determined by excluding the amount of ubiquitin or phosphoric acid is preferably used as the amount of the ubiquitinated or phosphorylated marker for depression. When a comparison is made between the ratio of a subject and the ratio of a healthy subject, the units of the ratios are preferably the same.

<Method Using Novel Marker Associated with Depression>

The marker for depression can be used for the diagnosis of depression.

The marker for depression can be used for an assay assisting the diagnosis of depression.

For example, the assay may include a step of examining the expression level of the marker for depression. According to the findings obtained by the present inventors, the expression level of the marker for depression is higher in patients with depression than in healthy subjects. Therefore, information about the degree of expression level of the marker for depression is useful for assisting the diagnosis of depression.

Further, the assay may include, for example, a step of examining the ubiquitination and/or phosphorylation of the marker for depression. According to the findings obtained by the present inventors, it is considered that the marker for depression is ubiquitinated and then degraded by proteasome. Further, it is considered that the activity of the marker for depression is reduced by transfer from the cell membrane surface into the cell caused by phosphorylation. That is, it is considered that when the level of ubiquitination and/or phosphorylation of the marker for depression is low, the amount of molecules of a monoamine such as noradrenaline in the synaptic cleft is insufficient. For this reason, information about the level of ubiquitination and/or phosphorylation of the marker for depression is useful for assisting the diagnosis of depression.

The marker for depression can be used in a method for determining depression. The method for determining depression does not include medical practices.

The method for determining depression includes a step of examining the expression level of the marker for depression in a blood sample collected from a subject.

In the present invention, a person who provides a blood sample to be subjected to the method for determining depression is referred to as a subject. The subject is not necessarily limited to a person diagnosed with depression by a doctor.

The blood sample is not particularly limited as long as it contains the marker for depression. As described above, it has been reported that there is a relationship between the expression of the noradrenaline transporter in the central nervous system and the expression of the noradrenaline transporter in leukocytes including lymphocytes and that a change in the noradrenaline transporter in platelets or leukocytes including lymphocytes is associated with the function of the noradrenaline transporter in the central nervous system and psychological symptoms such as depression. Therefore, the blood of a subject is suitable as a sample. The blood sample may be either arterial blood or venous blood, and may be collected from any site. For example, the blood sample is preferably peripheral blood or spinal fluid, and is more preferably peripheral blood. The blood sample can be collected from a subject according to a known ordinary method. Further, the blood sample can be stored according to a known ordinary method.

The blood sample may further be fractionated. More specifically, the marker for depression in a sample fractionated from the blood sample may be analyzed. In the method for determining depression, the sample may be, for example, a platelet fraction or a leukocyte fraction containing lymphocytes, preferably a platelet fraction or a lymphocyte fraction.

The sample such as a blood sample may be a composition containing the sample. The sample can be appropriately selected depending on an analysis method exemplified below.

The amount of the sample used in the method for determining depression is not particularly limited as long as the amount of the marker for depression can be determined. The amount of the sample may be appropriately determined according to the number of steps of operation, a reagent used, the performance of device used, etc.

The method for determining depression may further include a step of comparing the expression level of the marker for depression in the blood sample collected from the subject with the expression level of the marker for depression obtained as a control from at least one healthy subject.

In the method for determining depression, the term “healthy subject” includes a person free of depression without limitation, but is preferably a person diagnosed as not having depression by a doctor, more preferably a person not exposed to psychological stress or a person not having another neurological or psychological disorder.

The expression level of the marker for depression as a control refers to the expression level of the marker for depression obtained from a healthy subject. A sample obtained from a healthy subject is not particularly limited, but is preferably a blood sample. It is sufficient to obtain the amount of the marker for depression from at least one healthy subject, but the accuracy of the method for determining depression can be expected to be improved by using, as a control, the average of the amounts of the marker for depression obtained from two or more healthy subjects.

Since a comparison is made between the amount of the marker for depression of a healthy subject and the amount of the marker for depression of a subject, a sample amount, a sample treatment method, and a method for measuring the marker for depression are preferably the same between the healthy subject and the subject.

Further, in the method for determining depression, the subject may be determined to have depression when the expression level of the marker for depression in the blood sample collected from the subject is at a higher level than the control as a result of the comparison.

The “higher level” is not particularly limited as long as the expression level of the marker for depression in the subject is higher than the control. However, it is preferred that there is a significant difference between the expression levels of the marker for depression in the subject and the control.

For example, when the noradrenaline transporter is selected as the marker for depression, a comparison of the expression level of the noradrenaline transporter is made between a subject and a healthy subject. The same applies to a case where the dopamine transporter is selected as the marker for depression.

Further, for example, when the noradrenaline transporter and the dopamine transporter are selected as the markers for depression, a comparison of the expression level of the noradrenaline transporter and a comparison of the expression level of the dopamine transporter are made. When the expression level of one of the markers for depression in a subject is at a higher level than a control as a result of the comparison, the subject may be determined to have depression. Preferably, the subject is determined to have depression when the expression levels of both the markers for depression are at a higher level than controls.

Hereinbelow, a method for determining depression will be described which focuses attention on the ubiquitination and/or phosphorylation of the marker for depression.

The method for determining depression may include a step of analyzing the following (1) and (2) in a blood sample collected from a subject:

(1) a noradrenaline transporter expression level; and

(2) a ratio of a ubiquitinated and/or phosphorylated noradrenaline transporter.

The method for determining depression may further include a step of comparing the analysis results of the following (3) and (4) in the subject with analysis results of the following (3) and (4) obtained as controls from at least one healthy subject:

(3) a noradrenaline transporter expression level; and

(4) a ratio of a ubiquitinated and/or phosphorylated noradrenaline transporter.

Further, in the method for determining depression, the subject may be determined to have depression when, as a result of the comparison, the noradrenaline transporter expression level obtained from the blood sample collected from the subject is at a higher level than the control, or the ratio of the ubiquitinated and/or phosphorylated noradrenaline transporter obtained from the blood sample collected from the subject is at a lower level than the control.

The comparison is made between the noradrenaline transporter expression level obtained from the subject and that obtained from the healthy subject and between the ratio of the ubiquitinated and/or phosphorylated noradrenaline transporter obtained from the subject and that obtained from the healthy subject.

In the method for determining depression using the ratio of the ubiquitinated and/or phosphorylated noradrenaline transporter, the blood sample is not particularly limited as long as it contains the ubiquitinated and/or phosphorylated marker for depression. As described above, the blood sample is a preferred sample.

Further, the method for determining depression preferably includes addition of a proteasome inhibitor to the sample so that the ubiquitinated noradrenaline transporter can be detected with high sensitivity.

When the noradrenaline transporter and the ubiquitinated and/or phosphorylated noradrenaline transporter are purified from the sample to determine the ratio of the ubiquitinated and/or phosphorylated noradrenaline transporter, a method for the purification is not particularly limited. However, the purification method is preferably one that maintains the ratio of the ubiquitinated and/or phosphorylated noradrenaline transporter in the blood sample. Examples of such a method that can be appropriately used include known methods such as fractionation using molecular weight or electric charge, including chromatography or electrophoresis, precipitation, centrifugation, salting out, immunoprecipitation, a method using a quartz oscillation biosensor, and a method using a ubiquitin- or noradrenaline transporter-specific binding material. Some quantitative methods may directly use the blood sample or may require only simple fractionation of the noradrenaline transporter and the ubiquitinated and/or phosphorylated noradrenaline transporter from the blood sample.

The ratio of the ubiquitinated and/or phosphorylated noradrenaline transporter as a control is the ratio of the ubiquitinated and/or phosphorylated noradrenaline transporter obtained from a healthy subject. A method for calculating the ratio of the ubiquitinated and/or phosphorylated noradrenaline transporter is preferably the same between a subject and a healthy subject.

The “lower level” is not particularly limited as long as the ratio of the ubiquitinated and/or phosphorylated noradrenaline transporter obtained from the blood sample of the subject is at least less than the ratio of the ubiquitinated and/or phosphorylated noradrenaline transporter as the control. However, the ratio of the ubiquitinated and/or phosphorylated noradrenaline transporter obtained from the blood sample of the subject is preferably lower than the control with significant difference. Depending on the selection of the above-described calculating formula or the like, there may be a case where even when being higher than the value of the control, the value of the ratio of the ubiquitinated and/or phosphorylated noradrenaline transporter obtained from the blood sample of the subject is judged to be at a “lower level”.

The subject can be determined to have depression when the noradrenaline transporter expression level obtained from the blood sample collected from the subject is at a higher level than the control or when the ratio of the ubiquitinated and/or phosphorylated noradrenaline transporter obtained from the blood sample collected from the subject is at a lower level than the control. It is also preferred that the subject is determined to have depression when these two requirements are satisfied.

According to another embodiment, another sample other than the blood sample may be used as long as the sample can be collected while the subject and the healthy subject can be kept healthy. Examples of such a sample include cerebrospinal fluid, lymph fluid, neurons or tissues derived and/or differentiated from any cells or tissues using a regenerative medical technique, and established cells such as lymphocytes. Alternatively, a visualization technique may be used.

The marker for depression can be used for screening of antidepressants. For example, the screening may include a step of examining a change in the amount of the marker for depression.

More specifically, a candidate ingredient is administered to a sample derived from a patient with depression and when the amount of the marker for depression is lower than before administration, the candidate ingredient can be considered to be useful as an antidepressant. Alternatively, a candidate ingredient is administered and when the ubiquitination and/or phosphorylation ratio of the marker for depression is higher than before administration, the candidate ingredient can be considered to be useful as an antidepressant.

Examples of an object (sample for screening) to which a candidate ingredient is given include the above-described blood sample and an animal model.

A screening test is preferably performed in duplicate or more. However, when a candidate ingredient is confirmed to have the effect of improving depression in at least a singlicate test, there is a possibility that the candidate ingredient is useful as an antidepressant.

<Kit>

A kit according to the present invention includes at least a detector of the marker for depression. The kit preferably further includes one or more selected from a ubiquitinated protein collector and a phosphorylated protein collector. The kit more preferably further includes a proteasome inhibitor.

An example of the detector of the marker for depression includes an anti-depression marker antibody.

The anti-depression marker antibody to be used may be either a monoclonal antibody or a polyclonal antibody. Alternatively, an artificially-created antibody fragment may also be used. The anti-depression marker antibody to be used may be an appropriate commercial product.

Examples of the anti-depression marker antibody include an anti-noradrenaline transporter antibody and an anti-dopamine transporter antibody.

Examples of the anti-noradrenaline transporter antibody include Anti-Noradrenaline transporter antibody (abcam: Catalog No. ab41559), Anti-Norepinephrine Transporter Antibody (Millipore Corporation: Catalog No. AB2234), and Norepinephrine Transporter Antibody (Rockland: Catalog No. 200-301-D97).

Examples of the anti-dopamine transporter antibody include Anti-Dopamine transporter antibody (abcam: Catalog No. ab5990), Anti-Dopamine transporter, N-terminus (Millipore Corporation: Catalog No. MAB369), Anti-Dopamine transporter, C-terminus (Millipore Corporation: Catalog No. AB 1766), and Anti-Dopamine transporter (N-terminal) antibody (Sigma-Aldrich: Catalog No. D6944).

The ubiquitinated protein collector is not particularly limited as long as it can identify ubiquitin and the identified protein can be collected. Examples of the ubiquitinated protein collector include antibodies and beads for collecting a protein combined with ubiquitin. The ubiquitinated protein collector may be composed of a single member or a combination of two or more members, and may be appropriately selected. The ubiquitinated protein collector to be used may be an appropriate commercial product.

The phosphorylated protein collector is not particularly limited as long as it can identify a phosphorylated protein and the identified protein can be collected. Examples of such a phosphorylated protein collector include antibodies such as anti-phosphorylated threonine, anti-phosphorylated serine, and anti-phosphorylated tyrosine and beads for collecting a phosphorylated protein.

Preferred examples of the proteasome inhibitor include MG-132 and lactacystin.

The order in which the materials included in the kit are used is not particularly limited, and the marker for depression can be detected using these materials. Further, the marker for depression and the ubiquitinated and/or phosphorylated marker for depression can be discriminated. These materials may be used in combination with another material.

The kit is suitable for analyzing the amount of the marker for depression contained in a sample. The analysis of the amount is a concept that includes, without limitation, measurement of the absolute amount of the marker for depression in a sample and measurement of the relative amount of the marker for depression in a sample and also includes a comparison with specific one or more other components. The kit is preferably suitable for analyzing the ratio between the marker for depression and the ubiquitinated and phosphorylated marker for depression in a sample.

The sample analyzed with the kit is not particularly limited as long as it contains the marker for depression. Examples of the sample include, but are not limited to, animals, plants, microorganisms, and objects obtained by the application of a regenerative medical technique. Further, the sample may be appropriately selected from, without limitation, a cell, a tissue, an organ, and an individual. The sample is preferably a sample obtained from an animal, more preferably an animal body fluid or a homogenate of an animal tissue or organ, even more preferably blood, even more preferably a platelet fraction fractionated from blood or a leukocyte fraction containing lymphocytes, particularly preferably a platelet fraction or a lymphocyte fraction. The sample can be appropriately obtained by a known ordinary method.

The kit is preferably used for the diagnosis of depression. Further, the kit is preferably used also for carrying out the above-described method using a novel marker associated with depression. The kit is preferably used also for the diagnosis of another mental disorder such as autism or Asperger's syndrome.

The kit may further include an object suitable for a method for analyzing the marker for depression. Preferred examples of the analysis method include the above-described quantitative methods. Examples of the object that may be included in the kit include various antibodies, enzymes, buffers, salts, media, materials for culture such as culture sheets, stabilizers, antiseptics, transformed cells, vectors for transformation, primers, probes, gene fragments, nucleic acids such as siRNA and shRNA, markers, appropriate labeling materials such as radioactive materials, fluorescent materials, and dyes, and containers such as reaction plates.

### EXAMPLES

Hereinbelow, examples of the present invention will be described. The technical scope of the present invention is not limited to the following examples.

(A) Methods and Materials

<(1) Preparation of MAGE-D1 (Melanoma Antigen Gene-D1) Gene-Deficient Mice>

A targeting vector (Stratagene: pBlueScript) for homologous recombination between a MAGE-D1 gene (Gene bank: NM_{—}019791.2) exon and a drug (G418)-resistant gene (GenBank: U00004.1) was electroporated into embryo stem (ES) cells derived from 129Svj mice (supplied from National Center for Geriatrics and Gerontology), and drug-resistant colonies were selected. A homologous recombinant was identified from the selected drug-resistant colonies by Southern blotting. The identified target homologous recombinant ES cell clone was injected into C57BL/6J mouse blastocyst-stage embryos to generate chimeric mice. The chimeric mice were mated with wild-type C57BL/6J mice to generate F1 heterozygous MAGE-D1 gene-deficient mice. The heterozygous MAGE-D1 gene-deficient mice were mated with wild-type C57BL/6J mice up to the F10 generation. The mice having a 99.9% C57BL/6J genetic background were used (When mating with a C57BL/6J mouse is performed once, about half of the genes of a newborn mouse are derived from the C57BL/6J mouse used for mating. That is, it is considered that when mating with a C57BL/6J mouse is performed n times, about [1−(½)ⁿ⁺¹]×100% of genes are derived from the C57BL/6J mouse.) The mice having such a genetic background were defined as MAGE-D1 gene-deficient mice (hereinafter, also referred to as model mice) and used in the following tests.

<(2) Evaluation of Depression-Like Behavior by Forced Swim Test>

Experimental Equipment and Procedure: A water bath (diameter 15 cm×height 20 cm) filled with water (water temperature: about 22° C., depth: 13 cm) was used as experimental equipment. A wild-type C57BL/6J mouse as a control or a model mouse was placed in the water bath, and just after that, immobility time was measured by Scanet MV-10 AQ (Brain Science Idea, Osaka, Japan) every 1 minute for 10 minutes.

Thirty minutes before the test, 10 mg/kg of desipramine (Sigma, St. Louis, Mo.) or 3 mg/kg of atomoxetine (LKT Laboratories, Inc, MN) dissolved in normal saline was intraperitoneally administered. As a control, the solvent was intraperitoneally administered.

<(3) Evaluation of Noradrenaline Contents in Brain Tissues by High Performance Liquid Chromatography>

A wild-type C57BL/6J mouse or a model mouse was decapitated, and brain areas such as the frontal cortex, hippocampus, amygdala, hypothalamus, nucleus accumbens, and corpus striatum were excised while cooled with ice and stored at −80° C. until use. An internal reference material (Isoproterenol) was added to each of the brain areas, and each of the brain areas was ultrasonically homogenized in the presence of 0.2M PCA (perchronic acid) and deproteinized. The homogenate was centrifuged (20,000 g, 15 min, 1° C.) to collect a supernatant, and the supernatant was pH-adjusted with sodium acetate and filtered to obtain a sample. The amount of noradrenaline contained in the sample was measured by HPLC-ECD (EICOM Corp, Kyoto). A separation column (EICOMPAK SC-SODS, EICOM Corp) was used for analysis, and an electrochemical detector equipped with a graphite electrode (WE-3G) as a working electrode was used for detection, and the preset applied voltage of the detector was +400 mV vs Ag/AgCl.

<(4) Evaluation of Noradrenaline Release by In Vivo Microdialysis>

A mouse under anesthesia with pentobarbital sodium (50 mg/kg, i.p.) was fixed in a stereotaxic instrument, and a guide cannula (AG-6, EICOM Corp., Kyoto, Japan) was inserted into the frontal cortex (rostral side from the bregma on the skull: 1.7 mm, right side: +1.0 mm, depth: −1.5 mm) at an angle of 15° with reference to a brain atlas (Franklin and Paxinos, 1997). The guide cannula was fixed on the skull with dental cement (SHOFU Inc., Kyoto, Japan).

On the day following the surgery, a dialysis probe (A-I-6-1, 1 mm membrane length, EICOM Corp.) was inserted into the frontal cortex of the mouse through the guide cannula, and the mouse was placed in an acrylic case (30 cm×30 cm×35 cm) and allowed to freely move. Ringer's solution (NaCl: 147 mM, KCl: 4 mM, CaCl₂: 2.3 mM) was perfused into the probe at a flow rate of 1.0 μL/min. The perfusate was collected every 10 minutes, and the noradrenaline content of the collected perfusate was quantitatively determined by high performance liquid chromatography (HTEC-500, EICOM Corp.). As a mobile phase, a 99% (v/v) 0.1 M sodium phosphate buffer (pH 6.0) containing 1% (v/v) methanol, sodium decanesulfonate (SDS, 500 mg/L), and EDTA.2Na (50 mg/L) was used, and the mobile phase was allowed to flow at a flow rate of 500 μL/min. A separation column (EICOMPAK PP-ODS, 30×4.6 mm phi, EICOM Corp.) and a precolumn (EICOM PREPAKSET CA-ODS, EICOM Corp.) were used for analysis, an electrochemical detector equipped with a graphite electrode (WE-3G) as a working electrode was used for detection, and the preset applied voltage of the detector was +400 mV vs Ag/AgCl.

The time schedule was as follows: after the amount of released extracellular noradrenaline became stable (−60 mM to 0 min), high-potassium Ringer's solution (NaCl: 101 mM, KCl: 50 mM, CaCl₂: 2.3 mM) was perfused into the probe for 20 minutes (0 to 20 min), and then the perfusate was changed back to the Ringer's solution, and the amount of released extracellular noradrenaline was measured up to 3 hours.

<(5) Evaluation of Expression Level of Noradrenaline Transporter Protein by Western Blotting>

A brain sample or cells was ultrasonically broken by a sonicator at 4° C. in a lysis buffer (20 mM Tris-HCl, 150 mM NaCl, 50 mM NaF, 1 mM EDTA, 1 mM EGTA, 1% (w/v) Triton X-100, 1 mM sodium orthovanadate, 0.1% (w/v) SDS, 1% (w/v) sodium deoxycholate, 0.5 mM dithiothreitol, 10 mM sodium pyrophosphate decahydrate, 1 mM phenylmethylsulfonyl fluoride, 10 μg/mL aprotinin, 10 μg/mL leupeptin, and 10 μg/mL pepstatin (pH 7.4)) to obtain a homogenate. The homogenate was centrifuged at 4° C. and 13000×g for 20 minutes, and the thus obtained supernatant was used. A sample buffer (0.125 M Tris-HCl (pH 6.8), 2% (w/v) SDS, 5% (w/v) glycerol, 0.002% (w/v) bromphenol blue, and 5% (w/v) 2-mercaptoethanol) was added to each supernatant sample whose protein content had been adjusted, and the supernatant sample was boiled at 95° C. for 5 minutes. Then, protein (20 μg) was electrophoresed using a 10% polyacrylamide gel and transferred to a polyvinylidene difluoride (PVDF) membrane (Millipore Corporation, Billerica, Mass., USA), and blocked by adding Detector Block Kit (Kirkegaard and Perry Laboratories, Gaithersburg, Md., USA). A primary antibody (anti-NAT) against noradrenaline transporter protein (Abcam, Cambridge, Mass.) was added to the PVDF membrane, the PVDF membrane was allowed to stand overnight in a refrigerator (4° C.), a secondary antibody (HRP-conjugated anti-rabbit IgG) (Kirkegaard and Perry Laboratories) was added, and the PVDF membrane was allowed to stand at room temperature for 3 hours. Light emitted from an immunocomplex was detected using ECL (GE Healthcare Biosciences, Piscataway, N.J., USA) that is a detection reagent for Western blotting, and the expression level of noradrenaline transporter protein was calculated by the image analysis of emission intensity.

Then, in order to examine the expression level of β-actin protein that is an endogenous reference material, stripping was performed, a primary antibody (anti-β-actin) (Santa Cruz Biotechnology, Santa Cruz, Calif.) was added, and incubation was performed. The obtained band of noradrenaline transporter protein was corrected by the band of β-actin protein, and the expression level of noradrenaline transporter protein was expressed as a percentage (%) relative to that of a control group.

<(6) Evaluation of Expression Level of Ubiquitinated Noradrenaline Transporter Protein>

According to the procedure described in the above A(5), ubiquitinated protein was isolated using UbiQapture-Q kit (Enzo Life Sciences International, Inc, Plymouth Meeting, Pa.) from the homogenate obtained by homogenization in a lysis buffer. Ubiquitinated noradrenaline transporter protein was detected by performing Western blotting under the same conditions as in the above A(5).

<(7) Quantitative Evaluation of Noradrenaline Transporter mRNA>

Total RNA was extracted from the frontal cortex of a wild-type C57BL/6J mouse as a control and a model mouse, and cDNA synthesized using a reverse transcriptase was used as a template for real-time RT-PCR. The expression level of mRNA was quantitatively determined by a Taqman probe method. For the noradrenaline transporter gene, primers of SEQ ID Nos. 1 and 2 and a Taqman probe of SEQ ID No. 3 shown below were used.

SEQ ID No. 1:
5′-GAGCAGTGGGATCCATGACATC-3′
SEQ ID No. 2:
5′-CCAGAGGCTGAAATACAAGACAAG-3′
SEQ ID No. 3:
5′-ACGACCATCAGGCAGAGCAGCAGC-3′

For β-actin used as an internal reference, primers of SEQ ID Nos. 4 and 5 and a Taqman probe of SEQ ID No. 6 shown below were used.

SEQ ID No. 4:
5′-GGGCTATGCTCTCCCTCACG-3′
SEQ ID No. 5:
5′-GTCACGCACGATTTCCCTCTC-3′
SEQ ID No. 6:
5′-CCTGCGTCTGGACCTGGCTGGC-3′

It is to be noted that if the sequence described in Examples does not agree with that described in the sequence list, the sequence described in Examples takes priority.

<(8) Preparation of Established Lymphocytes from Blood of Healthy Subjects and Patients with Depression>

Objects were healthy subjects and patients with depression responsive to an antidepressant, fluvoxamine that is a selective serotonin reuptake inhibitor. The patients with depression were those who were diagnosed with depression by a doctor according to Hamilton Depression Scale (HAM-D). The healthy subjects were those who were diagnosed as healthy by a doctor.

Blood was aseptically collected from each of the objects and two-fold diluted with sterilized normal saline. Then, the diluted blood was gently layered on 3.5 mL of Ficoll-Paque (GE Healthcare, Uppsala, Sweden) contained in a tube, and the tube was centrifuged at 600×g for 30 minutes. Then, a white mononuclear cell layer observed as an intermediate part of the centrifuged sample and a Ficoll-Paque layer just below the mononuclear cell layer were collected. Then, the collected sample was suspended in normal saline and centrifuged at 400×g for 30 minutes. Then, a sediment obtained by the centrifugation was mixed with normal saline, and the mixture was centrifuged at 240×g for 5 minutes. Then, a sediment obtained by the centrifugation was mixed with a liquid medium (RPMI 1640), and the mixture was again centrifuged at 240×g for 5 minutes. Then, a sediment obtained by the centrifugation was regarded as a lymphocyte fraction and mixed with RPMI 1640 containing 20% fetal bovine serum, 20% culture supernatant of Epstein-Barr virus-releasing cell line (B95-8), and 2 μg/mL cyclosporin A, and the mixture was cultured for one week or more. Lymphocytes that proliferated were regarded as established lymphocytes and subcultured using RPMI 1640 containing 10% fetal bovine serum. The expression level of noradrenaline transporter protein contained in established lymphocytes prepared from the blood of each of the objects was evaluated in the same manner as in the above A(5).

(B) Results

<MAGE-D1 Gene-Deficient Mouse>

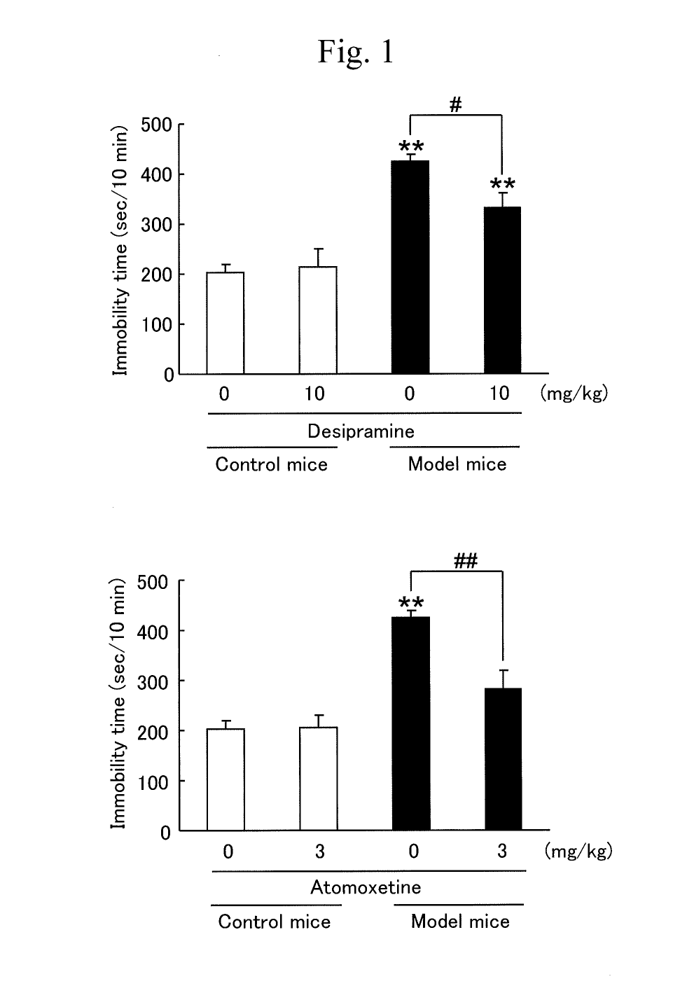

In Examples, MAGE-D1 gene-deficient mice were used as animal models. The forced swim test was used for evaluating depression-like behavior. In the forced swim test, a mouse is placed in a narrow cylinder filled with water so as to recognize the impossibility of escape therefrom, and the time that the mouse floats on water without moving is evaluated as decreased motivation. The immobility time of model mice in the forced swim test was increased, that is, the model mice displayed decreased motivation. Such an increase in immobility time was significantly reduced by both desipramine and atomoxetine (FIG. 1). Each administration test was performed in triplicate, and therefore graphs in FIG. 1 show average values. In the desipramine administration test, the average values of immobility time of control mice were 203 seconds at 0 mg/kg and 215 seconds at 10 mg/kg, and the average values of immobility time of model mice were 425 seconds at 0 mg/kg and 334 seconds at 10 mg/kg. In the atomoxetine administration test, the average values of immobility time of control mice were 203 seconds at 0 mg/kg and 205 seconds at 3 mg/kg, and the average values of immobility time of model mice were 425 seconds at 0 mg/kg and 283 seconds at 3 mg/kg.

It was suggested that the model mice exhibited depression-like behavior strongly responsive to a human antidepressant having a noradrenaline transporter inhibitory effect. Further, it was suggested that the model mice exhibited depression-like behavior strongly responsive also to a human anti-attention-deficit hyperactivity disorder medication having a noradrenaline transporter inhibitory effect. The depression-like behavior of the model mice is relieved by a human antidepressant, and therefore it is reasonably assumed that the effect observed in the model mice is observed also in humans. Further, it is reasonably assumed that in the mechanism of depression, the model mice and humans have something in common as for the noradrenalin transporter.

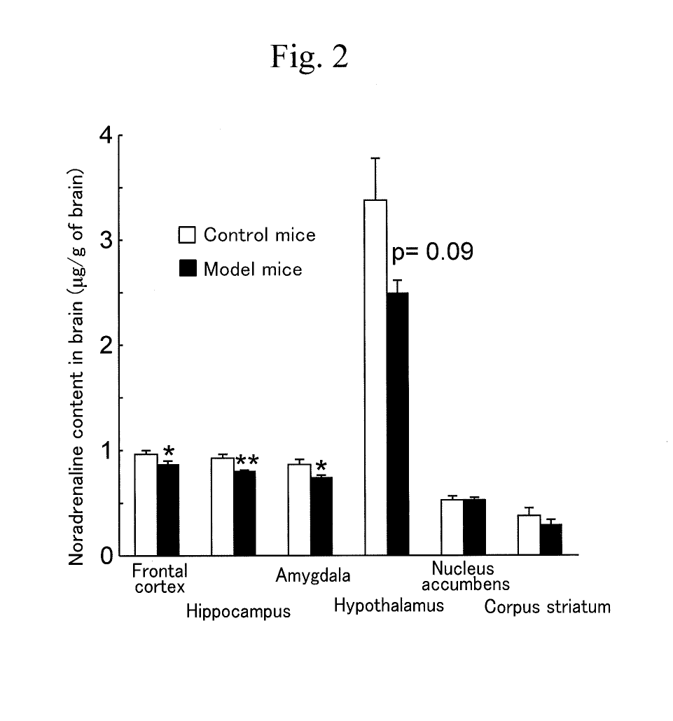

In the pathophysiology of depression, the monoamine hypothesis is known. Particularly, the noradrenergic neuronal system as well as the serotonergic neuronal system has been attracting attention as a target for antidepressants. The noradrenaline contents of brain tissues of model mice were evaluated by high-performance liquid chromatography measurement. The noradrenaline contents of the frontal cortex, the hippocampus, and the amygdala were significantly lower in model mice than in control mice (FIG. 2).

The test using a model mouse and the test using a control mouse were performed in decuplicate, and therefore a graph in FIG. 2 shows average values. The average noradrenaline content of the frontal cortex of control mice was 967 ng/g and that of model mice was 856 ng/g. The average noradrenaline content of the hippocampus of the control mice was 921 ng/g and that of the model mice was 795 ng/g. The average noradrenaline content of the amygdala of the control mice was 866 ng/g and that of the model mice was 740 ng/g. The average noradrenaline content of the hypothalamus of the control mice was 3378 ng/g and that of the model mice was 2492 ng/g. The average noradrenaline content of the nucleus accumbens of the control mice was 521 ng/g and that of the model mice was 520 ng/g. The average noradrenaline content of the corpus striatum of the control mice was 374 ng/g and that of the model mice was 282 ng/g.

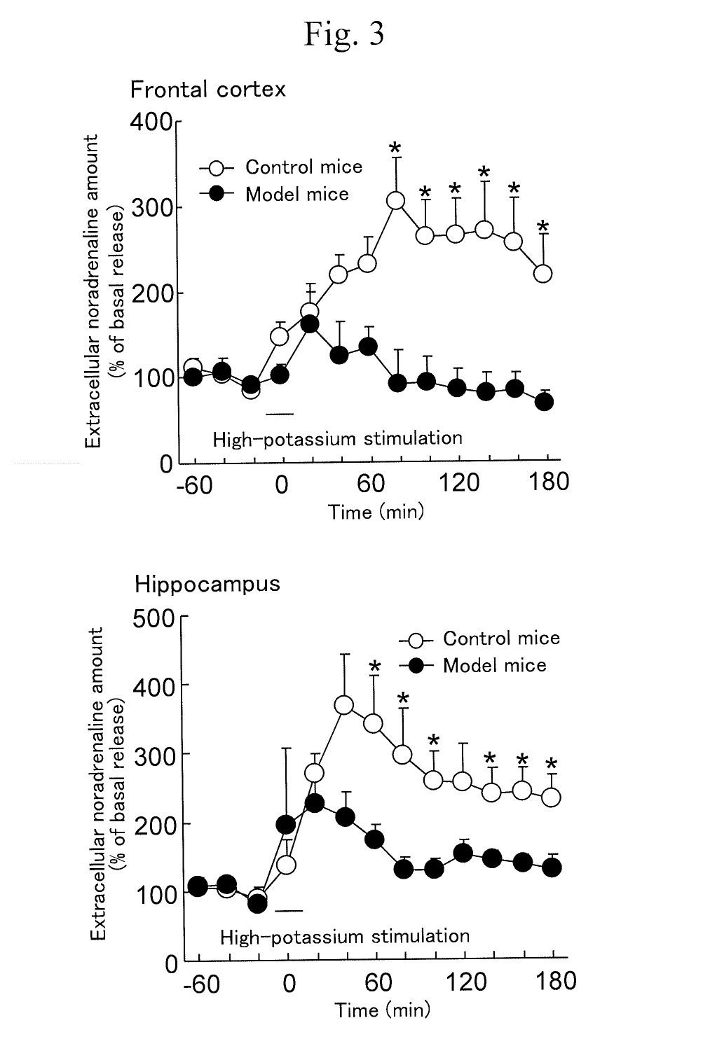

Further, the function of the noradrenergic nervous system in the brain of a model mouse was evaluated by brain microdialysis. An increase in the amount of released extracellular noradrenaline in the frontal cortex and the hippocampus caused by high-potassium stimulation was significantly reduced in model mice than in control mice (FIG. 3).

The test using a model mouse and the test using a control mouse were performed in quadruplicate to quintuplicate, and therefore graphs in FIG. 3 show average values. The amount of basal release is the average noradrenaline concentration of three samples obtained by collecting a perfusate, flowing from the brain of a mouse at a flow rate of 1 μL/min, for 10 minutes three times from −30 min to 0 min. The amount of basal release in the frontal cortex of model mice was 1.08±0.32 pmol/10 μL/10 min and that in the frontal cortex of control mice was 0.87±0.27 pmol/10 μL/10 min, and the amount of basal release in the hippocampus of the model mice was 0.92±0.17 pmol/10 μL/10 min and that in the hippocampus of the control mice was 0.92±0.13 pmol/10 μL/10 min. In FIG. 3, the amount of extracellular noradrenaline is expressed as a ratio relative to the amount of basal release.

The average amounts of extracellular noradrenaline in the frontal cortex of the control mice were 147.3% after 0 min (just after application of high-potassium stimulation), 175.5% after 20 min, 218.2% after 40 min, 231.1% after 60 min, 304.7% after 80 min, 263.7% after 100 min, 264.4% after 120 min, 263.7% after 140 min, 255.5% after 160 min, and 216.6% after 180 min, and those of the model mice were 101.5% after 0 min, 160.2% after 20 min, 123.7% after 40 min, 134.7% after 60 min, 91.0% after 80 min, 92.4% after 100 min, 84.6% after 120 min, 79.8% after 140 min, 82.7% after 160 min, and 67.3% after 180 min. The average amounts of extracellular noradrenaline in the hippocampus of the control mice were 137.0% after 0 min (just after application of high-potassium stimulation), 269.4% after 20 min, 366.2% after 40 min, 339.4% after 60 min, 295.3% after 80 min, 257.4% after 100 min, 257.4% after 120 min, 238.0% after 140 min, 240.0% after 160 min, and 229.4% after 180 min, and those of the model mice were 195.2% after 0 min, 226.4% after 20 min, 205.5% after 40 min, 172.9% after 60 min, 128.7% after 80 min, 127.8% after 100 min, 151.4% after 120 min, 142.2% after 140 min, 136.2% after 160 min, and 128.1% after 180 min. It was suggested that the function of the noradrenergic nervous system was decreased in the model mice.

### Example 1

### Expression of Noradrenaline Transporter in MAGE-D1Gene-Deficient Mice

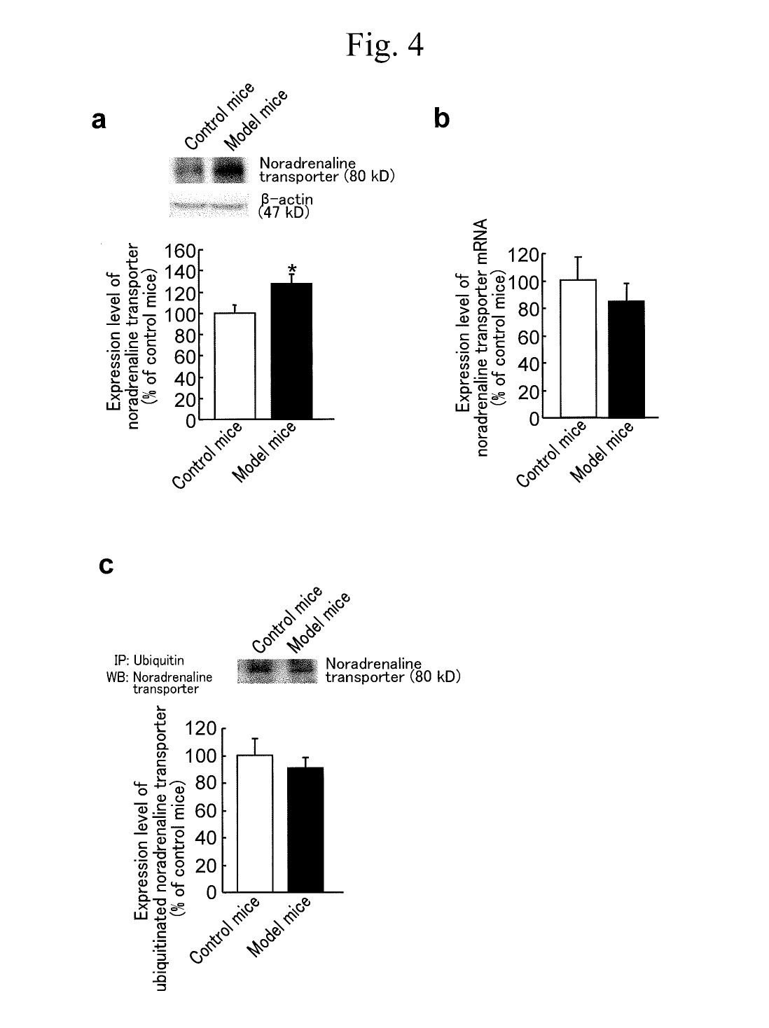

In order to find the cause of functional decline of the noradrenergic neuronal system in model mice, a change in the expression of noradrenaline transporter protein in the frontal cortex was examined using model mice and wild-type C57BL/6J mice as controls by Western blotting described in the above A(5). As a result, an increase in the amount of noradrenaline transporter protein (80 kD) in the frontal cortex of the model mice was observed (FIG. 4a: 124.2% with respect to the control mice). In order to examine the involvement of transcriptional control in such an increase in the amount of noradrenaline transporter protein, the amount of noradrenaline transporter mRNA was quantitatively determined by real-time PCR described in the above A(7). As a result, there was no difference in the amount of noradrenaline transporter mRNA between the model mice and the control mice (FIG. 4b: 84.8% with respect to the control mice). Further, the ubiquitination of noradrenaline transporter protein involved in the protein degradation of noradrenaline transporter protein was examined by the method described in the above A(6). As a result, there was no significant difference in the amount of ubiquitinated noradrenaline transporter protein between the model mice and the control mice (FIG. 4c: 91.0% with respect to the control mice). It was suggested that the amount of noradrenaline transporter protein in the model mice was increased by the absence of the MAGE-D1 gene. However, a change in the metabolism of noradrenaline transporter protein through the synthesis of noradrenaline transporter mRNA or ubiquitination was not observed.

Each of the tests whose results are shown in FIGS. 4a to 4c was performed using a model mouse and a control mouse in triplicate, and therefore the test results shown are average values.

### Example 2

### Expression Level of Noradrenaline Transporter in Established Lymphocytes Derived from Patients with Depression

In comparison with the expression level of the noradrenaline transporter in the established lymphocytes derived from the healthy subjects (the expression level is regarded as a control of the amount of noradrenaline transporter protein), the expression level of the noradrenaline transporter in the established lymphocytes derived from the antidepressant-responsive patients with depression was 130.1%, that is, was significantly increased. The test for a healthy subject and the test for a patient with depression were performed in sextuplicate, and therefore the test result shown is an average value.

The results of the tests showed that the expression level of the noradrenaline transporter in the established lymphocytes derived from the antidepressant-responsive patients with depression was increased.

In these experiments, a significance test was performed using one-way analysis of variance and Fisher's PLSD that is post-hoc analysis.

### INDUSTRIAL APPLICABILITY

According to the present invention, it is possible to provide a method and kit using a useful novel marker associated with depression.

1. A marker for depression comprising one or more selected from a noradrenaline transporter and a dopamine transporter.

2. A method for determining depression, comprising a step of examining an expression level of the marker for depression according to claim 1 in a blood sample collected from a subject.

3. The method for determining depression according to claim 2, further comprising a step of comparing the expression level of the marker for depression in the blood sample collected from the subject with an expression level of the marker for depression obtained as a control from at least one healthy subject.

4. The method for determining depression according to claim 3, wherein the subject is determined to have depression when the expression level of the marker for depression in the blood sample collected from the subject is at a higher level than the control as a result of the comparison.

5. A method for determining depression, comprising a step of analyzing the following (1) and (2) in a blood sample collected from a subject:
(1) a noradrenaline transporter expression level; and
(2) a ratio of a ubiquitinated and/or phosphorylated noradrenaline transporter.

6. The method for determining depression according to claim 5, further comprising a step of comparing the analysis results of the following (3) and (4) in the subject with analysis results of the following (3) and (4) obtained as controls from at least one healthy subject:
(3) a noradrenaline transporter expression level; and
(4) a ratio of a ubiquitinated and/or phosphorylated noradrenaline transporter.

7. The method for determining depression according to claim 6, wherein the subject is determined to have depression when the noradrenaline transporter expression level obtained from the blood sample collected from the subject is at a higher level than the control or when the ratio of the ubiquitinated and/or phosphorylated noradrenaline transporter obtained from the blood sample collected from the subject is at a lower level than the control as a result of the comparison.

8. A kit comprising a detector of the marker for depression according to claim 1.

9. The kit according to claim 8, further comprising one or more selected from a ubiquitinated protein collector and a phosphorylated protein collector.

10. An assay method for assisting diagnosis of depression, comprising a step of examining an expression level of the marker for depression according to claim 1.

11. An assay method for assisting diagnosis of depression, comprising a step of examining ubiquitination and/or phosphorylation of the marker for depression according to claim 1.

12. A screening method for antidepressants, comprising a step of examining a change in an expression level of the marker for depression according to claim 1.


SEQUENCE LISTING (6 sequences)

SEQ ID NO: 1 - DNA, 22 bp, Artificial
  NAT forward primer
gagcagtgggatccatgacatc

SEQ ID NO: 2 - DNA, 24 bp, Artificial
  NAT reverse primer
ccagaggctgaaatacaagacaag

SEQ ID NO: 3 - DNA, 24 bp, Artificial
  NAT Taqman probe
acgaccatcaggcagagcagcagc

SEQ ID NO: 4 - DNA, 20 bp, Artificial
  b-actin forward primer
gggctatgctctccctcacg

SEQ ID NO: 5 - DNA, 21 bp, Artificial
  b-actin reverse primer
gtcacgcacgatttccctctc

SEQ ID NO: 6 - DNA, 22 bp, Artificial
  b- actin Taqman probe
cctgcgtctggacctggctggc